(19) United States
(12) Patent Application Publication
(10) Pub. No.: US 2024/0166420 A1
(43) Pub. Date: May 23, 2024

(54) SYRINGE TRANSPORT TOOL SET, TRANSPORTATION METHOD OF SYRINGE, PACKAGING MEMBER, AND USE METHOD OF PACKAGING MEMBER

(71) Applicant: THREEBOND CO., LTD., Hachioji-shi, Tokyo (JP)

(72) Inventors: Misako HIROTA, Hachioji-shi, Tokyo (JP); Makoto KATO, Hachioji-shi, Tokyo (JP); Takashi NEMOTO, Hachioji-shi, Tokyo (JP)

(21) Appl. No.: 18/552,520

(22) PCT Filed: Mar. 25, 2022
(86) PCT No.: PCT/JP2022/014297

§ 371 Date: Sep. 26, 2023

(30) Foreign Application Priority Data
Apr. 15, 2021 (JP) 2021-069242

(51) Int. Cl. B65D 81/18; A61M 5/00; B65D 25/10; B65D 81/107; B65D 81/38

(52) U.S. Cl. CPC B65D 81/18; A61M 5/002; B65D 25/108; B65D 81/107; B65D 81/3825

(57) ABSTRACT

The present invention provides a syringe transport method, packaging member, a method for using the packaging member, and a syringe transport tool set that has: surrounding member that is arranged in a housing space in a packing container, encircles a syringe comprising a material storage container that stores a viscous material, and comprises buffer member that cushions against external force applied to the syringe during transportation of the syringe; and a cold storage container comprising a content that can be arranged between the syringe and the buffer member in the housing space and which cools the syringe and a content storage container that houses the content. The thermal conductivity of the buffer member is no more than 0.022W/m·K. The melting point of the content is no more than −30° C. The present invention uses a configuration other than vacuum heat insulation and prevents or suppresses excessive cooling of the viscous material.

### TECHNICAL FIELD

The present invention relates to a syringe transport tool set, a transportation method of a syringe, a packaging member, and a use method of the packaging member.

### BACKGROUND ART

There are various types of viscous materials, and examples thereof include a one-liquid type curable resin and a resin of a two-liquid curable type, and the like. Among them, some one-liquid curable resins undergo progression of curing even at room temperature, and there are some one-liquid curable resins need to be maintained at the state of 0° C. or lower in the stages from production to transportation and safekeeping, and the like.

As a technique for maintaining a temperature of 0° C. or lower during transportation or the like of the one-liquid curable resin, there is a technique for disposing dry ice inside a container using a heat insulating material (see Patent Literature 1). Another technique related to heat retention (refrigeration) of contents includes a technique including an inner box, an outer box, and a vacuum heat insulating material provided between the inner and outer boxes and covered with a film, in which the outer box and the vacuum heat insulating material are closely fixed to each other via a flexible member with an adhesive on both sides between the vacuum heat insulating material and the outer box (see Patent Literature 2). Here, the vacuum heat insulating material is a heat insulating material around which vacuum state is created.

### CITATION LIST

### Patent Literatures

Patent Literature 1: JP 4503457 B2
Patent Literature 2: JP H01-20631 A

### SUMMARY OF INVENTION

The present inventors have conducted intensive studies focusing on the point that when dry ice is stored in a container to be transported and a viscous material is transported as in Patent Literature 1, supercooling occurs and affects product performance. Specifically, there is a fear that the viscous material is crystallized by supercooling, or separation and sedimentation occur at the time of thawing of the viscous material after supercooling. Furthermore, the present inventors are concerned about the point that when a vacuum heat insulating material is used for a container to be transported as in Patent Literature 2, the weight of the transport container becomes relatively heavy and the amount of viscous material that can be transported at a time becomes relatively small, and the like.

Therefore, the present invention has been made to achieve the above-described problems, and an object of the present invention is to provide a syringe transport tool set, a transportation method of a syringe, a packaging member, and a use method of the packaging member that can suppress temperature fluctuation of a viscous material with respect to temperature fluctuation of outside air by using a configuration other than a vacuum heat insulating material, and prevent or suppress supercooling of the viscous material.

A syringe transport tool set according to one aspect of the present invention for solving the above problems includes a surrounding member and a cold storage agent. The surrounding member is installed in a housing space of a packing container, surrounds the syringe including a material storage container that houses the viscous material, and includes a buffer member that mitigates an external force applied to the syringe when the syringe is transported. The cold storage agent is installable between the syringe and the surrounding member in the housing space, and includes a content that refrigerates the syringe and a content storage container that houses the content. The buffer member has a thermal conductivity of 0.022 W/m·K or less, and the content is configured to have a melting point of −30° C. or less.

Furthermore, in a transportation method of a syringe according to one aspect of the present invention, a syringe is disposed in a housing space of the packing container. Then, the cold storage agent is disposed in the housing space so as to surround the syringe. Then, the surrounding member is disposed in the housing space so as to surround the syringe via the cold storage agent.

Furthermore, one aspect of the present invention is a packaging member that retains the syringe, and a use method of the packaging member.

### BRIEF DESCRIPTION OF DRAWINGS

FIG. 1 is a perspective view schematically illustrating a syringe transport tool set according to one embodiment of the present invention.

FIG. 2 is a plan view illustrating an inside of a packing container in the syringe transport tool set of FIG. 1.

FIG. 3 is a perspective view of a cold storage agent constituting the syringe transport tool set.

FIG. 4 is a front view illustrating the cold storage agent.

FIG. 5 is a perspective view illustrating a packaging member and a syringe housed in the syringe transport tool set.

FIG. 6 is a front view of FIG. 5.

FIG. 7 is a graph showing a temperature change of a viscous material with respect to a temporal change of an outside air temperature in Experiment 1.

FIG. 8 is a graph showing temperature changes when syringes housing a viscous material are set in packaging members according to an example and a comparative example and thawed in Experiment 2.

### DESCRIPTION OF EMBODIMENT

Hereinafter, modes for carrying out the present invention will be described in detail with reference to the drawings. The embodiment shown herein is exemplified to embody the technical idea of the present invention, and do not limit the present invention. Furthermore, other modes that can be implemented, examples, operation techniques, and the like that can be conceived by those skilled in the art or the like without departing from the gist of the present invention are all included in the scope and gist of the present invention and included in the invention described in the claims and the scope of equivalents thereof.

In addition, for convenience of illustration and ease of understanding, the drawings attached to the present specification may be schematically represented by changing a scale, a dimension ratio of length and width, a shape, and the like from actual ones as appropriate, but are merely examples, and do not limit the interpretation of the present invention.

Furthermore, In the following description, ordinal numerals such as “first” and “second” will be given to provide description, but are used for convenience and do not define any order unless otherwise specified.

Furthermore, in the following, a coordinate system is used in the drawings and the description thereof. X and Y are planar directions in which a packaging member 210, a buffer member 250, and a cold storage agent 300 are placed, and are referred to as a first direction X, a second direction Y, or a planar direction XY. Z corresponds to a height direction of a packing container 100 and the packaging member 210, and is referred to as a height direction Z.

### (Syringe)

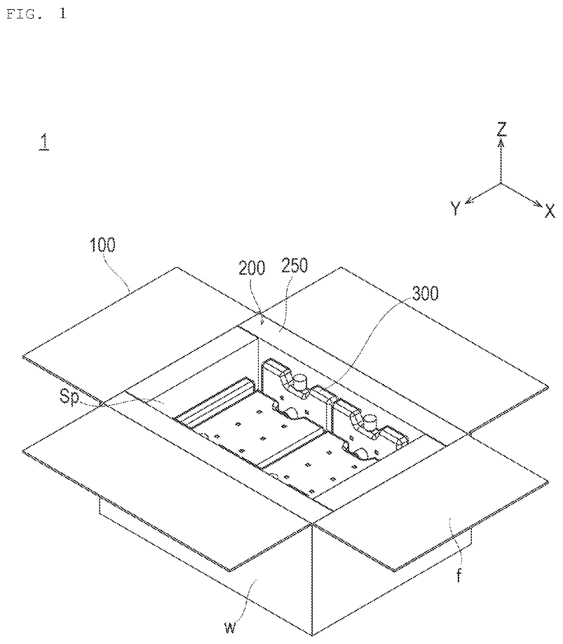

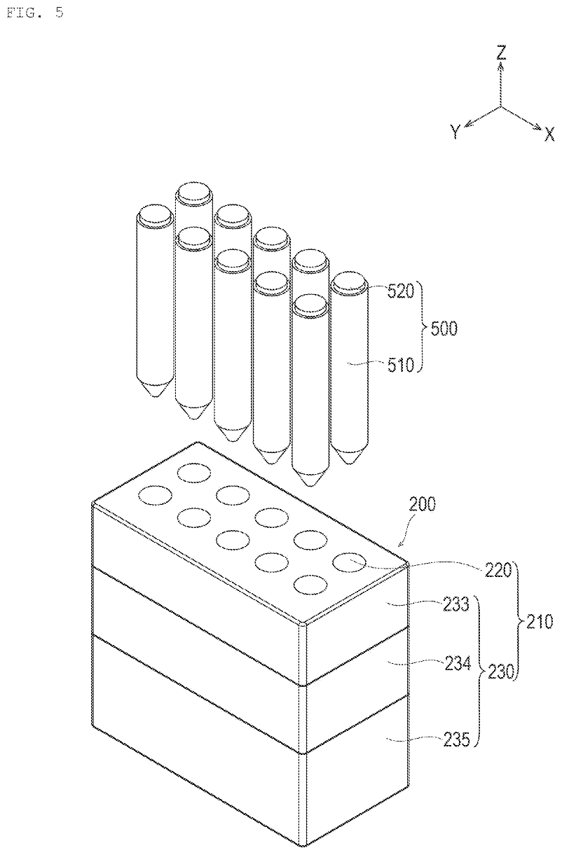

A syringe 500 is used by setting a viscous material in an application device such as a dispenser or the like. The syringe 500 includes, as illustrated in FIG. 5, a material storage container 510 and a lid portion 520. The syringe 500 can be installed in a housing space Sp (see FIG. 1) of the packing container 100.

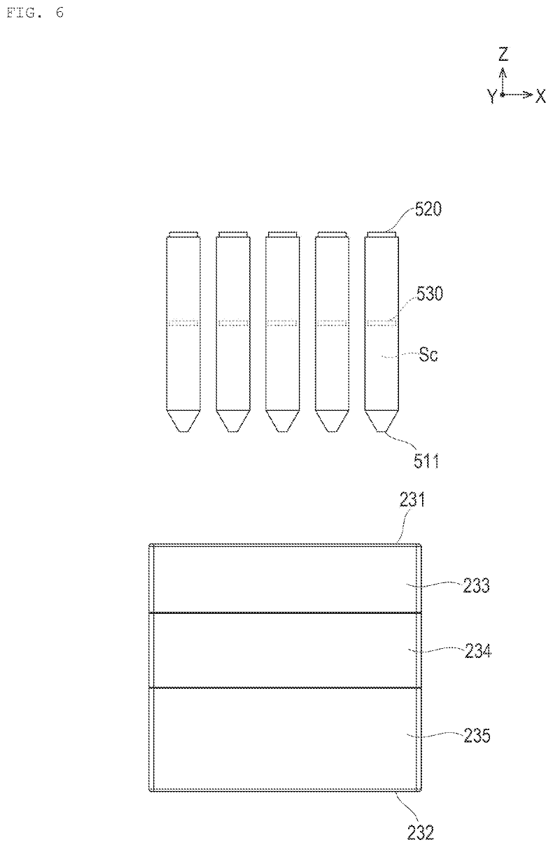

The material storage container 510 is configured to provide a semi-closed space Sc that houses the viscous material. The material storage container 510 includes, as illustrated in FIG. 6, an opening portion 511 through which the viscous material can be discharged from the semi-closed space Sc. The material storage container 510 can be provided with a plunger 530 at an intermediate portion in the height direction Z as illustrated in FIG. 6.

The plunger 530 can move the viscous material filled in the semi-closed space Sc in a sealed state toward the opening portion 511 by being movable in the height direction Z. The plunger 530 is provided, in the present embodiment, at the center in the height direction Z of the material storage container 510, but the position of the plunger is not limited to the center in the height direction Z as long as the viscous material can be stored in the semi-closed space Sc.

The material storage container 510 is configured such that a pointed shape or a curved surface shape is provided at the tip of the substantially cylindrical shape in the height direction Z. However, the specific shape is not limited to the above as long as a semi-closed space that houses the viscous material can be formed.

The lid portion 520 is attached near an end portion opposite to the opening portion 511 in the height direction Z in a state where the viscous material is housed in the semi-closed space Sc of the material storage container 510. The syringe 500 can install the material storage container 510 in the dispenser or the application device in a state where the viscous material is stored in the semi-closed space Sc of the material storage container 510 and the lid portion 520 is removed from the material storage container 510.

Examples of the viscous material transported by a syringe transport tool set 1 according to the present embodiment include, specifically, an adhesive, a sealant, a coating agent, a conductive adhesive, a thermally conductive resin, a flame-retardant resin, and the like, and among them, an adhesive, a sealant, a conductive adhesive, and a thermally conductive resin are preferable, and an adhesive and a conductive adhesive are exemplified as being particularly preferable, since the temperature fluctuation of the viscous material with respect to the temperature fluctuation of the outside air can be further suppressed.

The component of the viscous material is not particularly limited, and examples thereof include a urethane resin, an epoxy resin, an oxetane resin, a (meth) acrylic resin, a silicone resin, and the like, and among them, an epoxy resin, an oxetane resin, and a (meth) acrylic resin are preferable, and an epoxy resin is particularly preferable. By using more preferable components, it is possible to further suppress the temperature fluctuation of the viscous material with respect to the temperature fluctuation of the outside air.

The viscosity of the viscous material is not particularly limited, but is preferably in the range of 0.1 to 150 Pa·s, more preferably 1 to 75 Pa·s, and particularly preferably 5 to 30 Pa·s. Within the above range, the temperature fluctuation of the viscous material with respect to the temperature fluctuation of the outside air can be further suppressed. The measurement of the viscosity in the present invention is not particularly limited, but for example, 2.0 mL of the viscous material is weighed after stirring with a polytetrafluoroethylene rod, and the viscosity is measured using Brookfield (model number: DV-2+Pro) in a state where the temperature is set to 25° C. by a temperature control device. As measurement conditions, CPE-41 (3°×R2.4) was used for a cone rotor, the rotation speed was set to 10 rpm, and the viscosity after 3 minutes was set to “viscosity (Pa·s)”.

### (Syringe Transport Set)

The syringe transport tool set 1 includes, as illustrated in FIG. 1, the packing container 100, a surrounding member 200, and the cold storage agent 300. The syringe 500 transported by the syringe transport tool set 1 can be installed on the packaging member 210 in a state where the lid portion 520 is attached to the material storage container 510. Hereinafter, description will be made.

### (Packing Container)

As the packing container 100 illustrated in FIG. 1, a known cardboard or the like can be used. The packing container 100 can be configured by joining materials such as waste paper, pulp, or the like with glue or the like. Furthermore, the packing container 100 can form a closed space that stores the viscous material by maintaining flaps f provided continuously in both the upper and lower sides in the height direction Z of a side surface w in a state of being closed with an adhesive tape or the like in each of the upper and lower sides.

### (Surrounding Member)

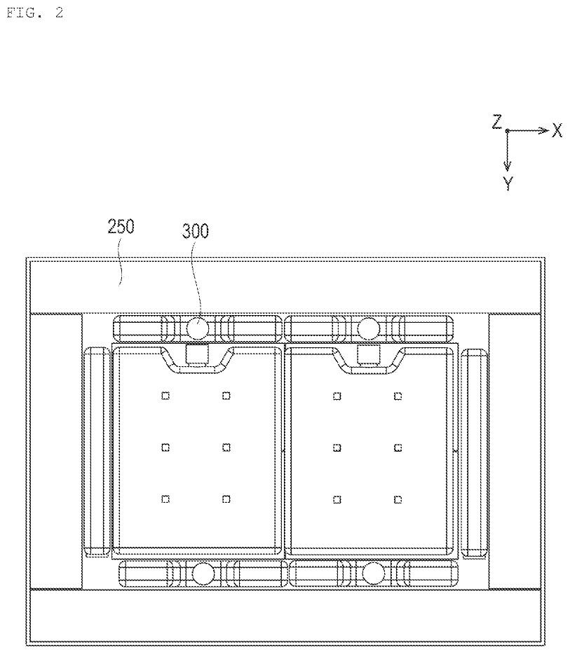

The surrounding member 200 is, as illustrated in FIG. 1, FIG. 2, installed in the housing space Sp of the packing container 100 and surrounds the syringe 500. The surrounding member includes the packaging member 210 capable of packaging at least a part of the syringe 500 in the present embodiment, and the buffer member 250 that mitigates an external force applied to the syringe 500 when the syringe 500 is transported.

### (Packaging Member)

The packaging member 210 is used to retain the syringe 500. The packaging member 210 includes, as illustrated in FIG. 5, an insertion portion 220 and a solid portion 230.

The insertion portion 220 is provided in the solid portion 230 and is configured to allow insertion of the syringe 500 in a state where the syringe 500 is erected. In the insertion portion 220, a cross section orthogonal to the longitudinal direction (height direction Z) in accordance with the shape of the syringe 500 is configured in a perfectly circular shape. However, the shape of the insertion portion is not limited to this as long as the syringe 500 can be retained, and the insertion portion may be configured by another circle other than a perfect circle such as an ellipse or the like, or a polygon.

The solid portion 230 is provided around the insertion portion 220 and is configured to form the insertion portion 220. The solid portion 230 is, as illustrated in FIG. 6, configured to be formed from a first end portion 231 where the insertion portion 220 is located to a second end portion 232 on the opposite side of the first end portion 231 in the direction in which the viscous material is inserted.

The solid portion 230 is configured to contain non-crosslinked foamed polyethylene. The linear expansion coefficient of the non-crosslinked foamed polyethylene constituting the solid portion 230 is 0.1 to 10×10⁻⁴ cm/cm·° C., and preferably 1 to 7×10⁻⁴ cm/cm·° C.

The solid portion 230 is, as illustrated in FIG. 5 in the present embodiment, configured in a layer shape so as to sequentially provide a first layer 233, a second layer 234, and a third layer 235 in the insertion direction (height direction Z) in which the syringe 500 is inserted. However, as long as the insertion portion can be formed, the number of layers is not limited thereto, and may be a single layer.

The solid portion 230 is configured such that the second end portion 232 becomes a flat surface so that the packaging member 210 can be easily installed as illustrated in FIG. 6.

### (Buffer Member)

The buffer member 250 mitigates an external force applied to the syringe 500 when the syringe 500 is transported. The buffer member 250 is configured to have a thermal conductivity of 0.022 W/m·K or less (JIS A9521). Note that the thermal conductivity is a value according to JIS A9521. As the buffer member 250, a building heat insulating material containing polystyrene or the like containing air bubbles can be used. The buffer member 250 is configured in a substantially rectangular parallelepiped shape in the present embodiment. However, the specific shape is not limited to a rectangular parallelepiped as long as the temperature of the viscous material can be easily maintained. The internal volume of the buffer member 250 is, for example, 318 mm×415 mm×330 mm.

By being configured as described above, the buffer member 250 can be made lighter than the vacuum heat insulating material, and can contribute to increasing the viscous material that can be transported at one time. The weight of the buffer member 250 can be set to about 4 kg while the vacuum heat insulating material having the same volume is about 13 kg. The weight of the buffer member 250 per one buffer member can be configured to be about 760 g as an example. The vacuum heat insulating material described above can be configured such that, for example, a glass wool core material is wrapped with a gas barrier film and sealed in a vacuum state.

### (Cold Storage Agent)

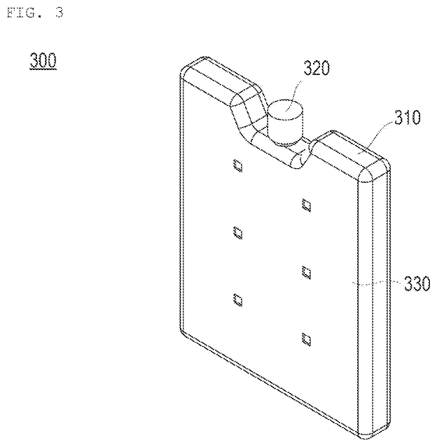

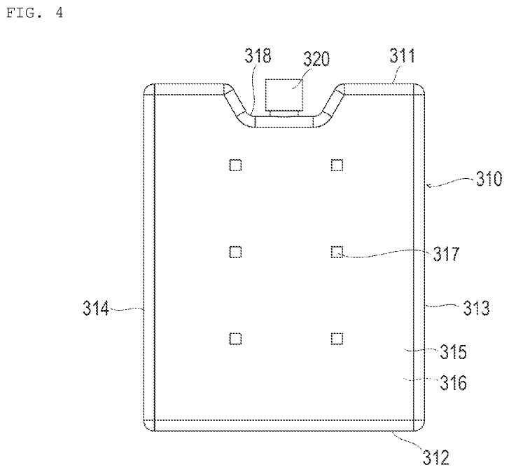

The cold storage agent 300 cools the syringe 500 housed in the packing container 100. The cold storage agent 300 includes, as illustrated in FIG. 3 and FIG. 4, a content storage container 310, a cap 320, and a content 330. The weight of the cold storage agent 300 per one cold storage agent can be configured to be, for example, about 1300 g.

The content storage container 310 is configured to be installable between the syringe 500 and the buffer member 250 in the housing space Sp of the packing container 100. The content storage container 310 is provided with a housing space capable of housing the content 330.

The content storage container 310 is configured in a shape such as a rectangular parallelepiped as an example so as to be housed in the packing container 100 in various postures. That is, surfaces 311 to 316 of the content storage container 310 are configured as flat surfaces. However, the shape of the content storage container 310 is an example, and the specific shape is not limited to a rectangular parallelepiped as long as being able to be housed in various postures.

The content storage container 310 is configured such that recessed portions 317 are provided on surfaces 311, 316 having a relatively large area among six surfaces 311 to 316 illustrated in FIG. 4. In addition, the content storage container 310 is configured such that a recessed portion 318 is provided in a part of the hexahedron such that the cap 320 to lid hardly protrudes from the content storage container 310 in a state where the content 330 which is a refrigerant is housed in the housing space. However, as long as the viscous material to be transported can be maintained at a predetermined temperature, the recessed portions 317, 318 may not be provided in the material storage container.

The material constituting the content storage container 310 is not particularly limited as long as the material does not deform so that the volume significantly changes during transportation or is difficult to deform, but examples thereof include polyethylene, polypropylene, polyethylene terephthalate, and the like. The content storage container 310 may adopt either a bag type that is relatively easily deformed by application of an external force or a hard type that is relatively hardly deformed and easily maintains a predetermined shape even when an external force is applied, but it is preferable to adopt the hard type from the viewpoint of difficulty in deformation as described above.

The content 330 cools the syringe 500 housed in the packing container 100. The content 330 is configured to have a melting point of −30° C. or lower. The lower limit value of the melting point of the content 330 is not particularly limited, but is preferably −50° C. The content 330 can be configured to include an aqueous solution such as an inorganic salt.

### (Transportation Method of Syringe)

Next, a transportation method of the syringe 500 using the syringe transport tool set according to the present embodiment will be described.

First, a packing container 100 such as cardboard or the like is prepared, and one of the flap portions (the lower side of the side surface w) of the packing container 100 is held a closed state by attaching an adhesive tape such as a packing tape or the like.

Next, the buffer member 250 is placed inside the side surface w of the packing container 100. The buffer member 250 can be installed close to the side surface w to such an extent that the side of the syringe 500 and the packaging member 210 to be housed can be surrounded.

After the buffer member 250 is installed on the side in the internal space of the packing container 100, the syringe 500 and the packaging member 210 as housed objects are disposed at substantially the center portion in the planar direction XY of the packing container 100. The syringe 500 and the packaging member 210 can be disposed in a state of forming a gap with the buffer member 250 in the first direction X or the second direction Y.

After the syringe 500 and the packaging member 210 are disposed in the internal space (housing space) of the packing container 100, the cold storage agent 300 is housed between the buffer member 250 and the syringe 500 and the packaging member 210. By disposing the buffer member 250 and the cold storage agent 300 between the syringe 500 and the packaging member 210 as contents, the syringe 500 can be transported in a state of being cooled to an extent that the syringe 500 does not become supercooled.

Next, the flaps f on the side opposite to the bottom surface of the packing container 100 (the upper side of the side surface w) is retained in a closed state with an adhesive tape or the like and transported toward the destination.

### (Use Method of Packaging Member)

Next, a use method of the packaging member 210 will be described. The packaging member 210 is taken out from the internal space of the packing container 100, and the container is thawed from −40° C. to room temperature in a state where the syringe 500 is inserted into the insertion portion 220. As a result, the temperature of the viscous material filled in the syringe 500 can be relatively slowly changed to room temperature to bring the viscous material in a usable.

As described above, the syringe transport tool set 1 according to the present embodiment includes the surrounding member 200 and the cold storage agent 300. The surrounding member 200 is installed in the housing space Sp of the packing container 100, and surrounds the syringe 500 including the material storage container 510 that houses the viscous material. The surrounding member 200 includes the buffer member 250 that mitigates an external force applied to the syringe 500 when the syringe 500 is transported.

The cold storage agent 300 can be installed between the syringe 500 and the buffer member 250 in the housing space Sp, and includes the content 330 that refrigerates the syringe 500 and the content storage container 310 that houses the content 330. The buffer member 250 has a thermal conductivity of 0.022 W/m·K or less, and the content 330 is configured to have a melting point of −30° C. or less.

When the dry ice is installed in the transport container and the viscous material is transported as described above, the viscous material is supercooled. On the other hand, when the vacuum heat insulating material is used for the transport container, the weight becomes relatively heavy, and the viscous material that can be transported at a time becomes relatively small. On the other hand, with a configuration as described above, it is possible to prevent or suppress supercooling of the viscous material by using a material other than the vacuum heat insulating material. Furthermore, by transporting the viscous material using the syringe transport tool set 1, it is possible to suppress temperature fluctuation of the viscous material with respect to temperature fluctuation of the outside air.

Furthermore, the content storage container 310 is configured such that the surfaces 311 to 316 have a flat surface. Therefore, the cold storage agent 300 can be installed in various postures with respect to the packing container 100, and the restriction caused by installing the cold storage agent 300 in the packing container 100 can be reduced to facilitate transportation of the syringe 500.

Furthermore, in the transportation method using the syringe transport tool set 1, the syringe 500 is disposed in the housing space Sp of the packing container 100, and the cold storage agent 300 is disposed in the housing space Sp so as to surround the syringe 500. Then, the buffer member 250 is disposed in the housing space Sp so as to surround the syringe 500 via the cold storage agent 300.

With such a configuration, it is possible to prevent or suppress a change in product performance of the viscous material housed in the syringe 500 during transportation due to supercooling, and to prevent crystallization, separation, and sedimentation of the viscous material due to supercooling.

Furthermore, the packaging member 210 includes the insertion portion 220 and the solid portion 230. The insertion portion 220 is configured to allow insertion of the syringe 500 in a state where the syringe 500 is erected. The solid portion 230 is provided around the insertion portion 220 and is configured to form the insertion portion 220.

The solid portion 230 is formed from the first end portion 231 on the side where the insertion portion 220 is located to the second end portion 232 on the opposite side of the first end portion 231 in the direction in which the viscous material is inserted into the insertion portion 220. The solid portion 230 is configured to contain non-crosslinked foamed polyethylene.

With such a configuration, it is possible to prevent a rapid temperature rise of the viscous material when the viscous material housed in the syringe 500 is thawed. Furthermore, it is possible to suppress air bubbles from entering the interface between the syringe 500 and the viscous material and to mitigate impact during transportation.

Furthermore, the solid portion 230 is formed in a layer shape in a direction in which the syringe 500 is inserted into the insertion portion 220. Therefore, the packaging member can be formed into a shape suitable for containers of any size, and the need for a mold can be eliminated, so that the manufacturability of the packaging member 210 can be improved or made favorable.

Furthermore, the solid portion 230 is configured to provide a flat surface on the second end portion 232. With such a configuration, the viscous material can be transported in a state where the packaging member 210 is stably placed on the placement surface during transportation.

Furthermore, in the use of the packaging member 210, the syringe 500 is configured to be thawed from −40° C. to room temperature in a state where the syringe 500 is inserted into the insertion portion 220 of the packaging member 210. With such a configuration, it is possible to prevent a rapid temperature rise of the viscous material housed in the syringe 500 and to prevent or suppress air bubbles from entering the interface between the syringe 500 and the resin.

### Experiment 1

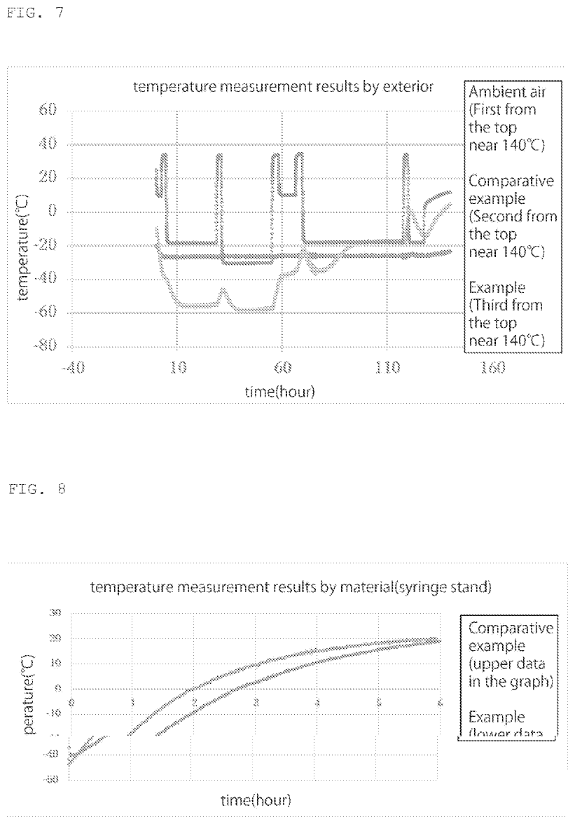

Hereinafter, an experiment related to transportation of a viscous material was performed, and thus will be described. FIG. 7 is a graph illustrating a temperature change of the viscous material with respect to a temporal change of an outside air temperature when the syringe housing the viscous material is transported using the transport tool sets according to an example and a comparative example in Experiment 1.

In Experiment 1, as a comparative example, a state was provided in which polystyrene foam, dry ice, and a viscous material housed in a syringe as a transportation target were installed in a cardboard packing container, and the packing container was installed in a thermostatic chamber in which the outside air temperature was set as described in FIG. 7 by a program for 140 hours. The melting point of the dry ice is −56.6° C. As polystyrene foam, one polystyrene foam having an outer size of 370 mm×496 mm×437 mm and a thermal conductivity of 0.04 W/m·K was used. As the dry ice, 14 pieces of dry ice having a weight of 500 g per one piece were used. As the syringe, a syringe made of polypropylene manufactured by Musashi Engineering, Inc. and having a capacity of 70 cc, a total length of 223.7 mm, an outer shape of φ 26.5 mm, and a flange outer shape of 45×30 mm was used.

Furthermore, as a transport tool set according to the example, a state was provided in which polystyrene foam (corresponding to buffer member, thermal conductivity is 0.022 W/m·K), a cold storage agent, and a viscous material housed in a syringe as a transportation target were installed in a cardboard packing container, and the packaging container was placed in a thermostatic bath for 140 hours. The melting point of the content of the cold storage agent is −30° C. The viscous material used is an epoxy resin.

The temperature of the viscous material housed in the syringe was measured in a state where the viscous material was installed in a thermostatic bath for 140 hours, and it was confirmed whether the measured temperature of the viscous material could be maintained within a range of −40° C. to −20° C., which did not affect or hardly affect the characteristics of the viscous material. Experimental results of the example and the comparative example are illustrated in FIG. 7.

In FIG. 7, the first line from the top at a temperature of 140° C. corresponds to the outside air (temperature), the second line from the top corresponds to the comparative example, and the third line from the top corresponds to the example. As can be seen from the graph, in the comparative example, it could be confirmed that the temperature was lower than −40° C. until 60 hours elapsed from the start, that is, so-called supercooling, and the temperature became higher than −20° C. after 120 hours elapsed.

On the other hand, it could be confirmed that the temperature could be maintained from −20° C. to −40° C. from the beginning to the end in the specification according to the example. That is, it could be confirmed that there was a high possibility that the product performance of the viscous material was affected during transportation in the specification according to the comparative example, while there was a high possibility that transportation could be performed without affecting the product performance of the viscous material in the specification according to the example. Note that, as the buffer member according to the example, since supercooling can be prevented even in the vacuum heat insulating material, it is conceived that the buffer material can be established even with thermal conductivity thereof being preferably 0.002 (more preferably 0.01) W/m·K or more, which is about the same as the thermal conductivity of the vacuum heat insulating material.

### Experiment 2

Next, the temperature change at the time of thawing of the viscous material using the packaging member was confirmed, and thus will be described.

In Experiment 2, the temperature change of the viscous material when the viscous material was thawed using two kinds of packaging members was confirmed. The viscous material used in the experiment is an epoxy resin, and specifications of the syringe are as follows: a capacity of 70 cc, a total length of 223.7 mm, an outer diameter φ of 26.5 mm, and an external dimension of an insertion portion of 45 mm×30 mm, manufactured by Musashi Engineering, Inc.

The packaging member according to the used comparative example is configured as illustrated in FIG. 5, and is a packaging member in which dimensions of length, width, and height are 120 mm×220 mm×220 mm, 10 syringes can be set, and the inside is a hollow paper packaging member. On the other hand, the packaging member according to the example is a packaging member in which dimensions of length, width, and height are 120 mm×220 mm×220 mm, 10 syringes can be set, the inside is solid, and non-crosslinked polyethylene (SUNTEC FOAM Q35) in which three layers are formed in the height direction is formed in an overlapping manner.

In Experiment 2, 10 syringes were set in the packaging members according to the comparative example and the example, and the temperature change of the viscous material when the ambient temperature was changed from −40° C. to 20° C. corresponding to room temperature was confirmed. The temperature was measured using a data logger in a thermostatic bath set at 20° C. with a thermocouple attached to the center of the syringe in the height direction. Furthermore, in this experiment, the viscous material was thawed by the packaging members according to the example and the comparative example, and it was confirmed whether or not the bead formed by the applied viscous material when applied from the dispenser was continuous. This is because, if air bubbles are generated at the time of thawing, it is considered that the bead cannot be continuously applied when being applied from the dispenser.

As the experimental results, as described above, the temperature changes of the comparative example and the example were confirmed, and whether air bubbles were generated in the viscous material such as the syringe or the like was visually confirmed in a state where the viscous material was housed in the syringe. Furthermore, application of the viscous material thawed using the packaging members according to the example and the comparative example was performed using a dispenser in which the tip shape of the discharge portion was a needle (30 G), and whether or not the formed bead shape was discontinued was visually confirmed. The above test is also called a discharge test or the like, and air bubbles inside the viscous material that cannot be visually confirmed by appearance are confirmed by directly blowing air into the syringe to discharge the entire amount of the viscous material. Hereinafter, a graph of temperature changes when the syringes housing the viscous material in the packaging members according to the comparative example and the example were thawed from −40° C. to room temperature is illustrated in FIG. 8. In FIG. 8, data located on the upper side corresponds to the comparative example, and data located on the lower side corresponds to the example.

It could be confirmed that the temperature change from −40° C. to the room temperature was rapid in the paper packaging member according to the comparative example as compared with the packaging member according to the example. Furthermore, in the packaging member according to the comparative example, air bubbles could be visually confirmed inside the syringe in the discharge test, whereas in the packaging member according to the example, air bubbles could not be confirmed inside the syringe.

In addition, it could be confirmed that in the application of the viscous material from the dispenser, the application breakage of the bead of the viscous material occurred in the specification according to the comparative example, whereas in the specification according to the example, the application breakage of the viscous material did not occur, and the bead was continuous. From the above, it could be confirmed that the viscous material thawed according to the specifications of the example could be attached to a dispenser or the like and maintained in a usable (dischargeable) state.

Note that the present application is based on JP application 2021-069242 filed on Apr. 15, 2021, the disclosure of which is incorporated herein by reference in its entirety.

### REFERENCE SIGNS LIST

100 packing container
200 surrounding member
210 packaging member
220 insertion portion
230 solid portion
231 first end portion
232 second end portion
233 first layer
234 second layer
235 third layer
250 buffer member
300 cold storage agent
310 content storage container
500 syringe
510 material storage container
520 lid portion

1. A syringe transport tool set comprising:
a surrounding member that is installed in a housing space of a packing container, surrounds a syringe including a material storage container that houses a viscous material, and includes a buffer member that mitigates an external force applied to the syringe when the syringe is transported; and
a cold storage agent that is installable between the syringe and the buffer member in the housing space and includes a content that refrigerates the syringe and a content storage container that houses the content,
wherein the buffer member has a thermal conductivity of 0.022 W/m·K or less, and
the content is configured to have a melting point of −30 degrees or less.

2. The syringe transport tool set according to claim 1, wherein the content storage container has a flat surface.

3. A transportation method of a syringe, comprising:
disposing the syringe in the housing space of the packing container according to claim 1;
disposing the cold storage agent in the housing space so as to surround the syringe; and
disposing the buffer member in the housing space so as to surround the syringe via the cold storage agent.

4. A packaging member used in the syringe transport tool set according to claim 1, wherein
the surrounding member includes a packaging member capable of packaging at least a part of the syringe and a buffer member that mitigates an external force applied to the syringe when the syringe is transported,
the packaging member includes an insertion portion that allows insertion of the syringe in a state where the syringe is erected, and a solid portion that is provided around the insertion portion and forms the insertion portion, and
the solid portion is formed from a first end portion on a side where the insertion portion is located to a second end portion on a side opposite to the first end portion in an insertion direction in which the viscous material is inserted into the insertion portion, and contains non-crosslinked foamed polyethylene.

5. The packaging member according to claim 4, wherein the solid portion is formed in a layer shape in the insertion direction.

6. The packaging member according to claim 4, wherein the solid portion is provided with a flat surface at the second end portion.

7. A use method of a packaging member comprising, thawing the syringe according to claim 4 from −40° C. to room temperature in a state where the syringe is inserted into the insertion portion.
